(19) United States
(12) Patent Application Publication
(10) Pub. No.: US 2026/0144919 A1
(43) Pub. Date: May 28, 2026

(54) FILTER DEVICE

(71) Applicant: TERUMO KABUSHIKI KAISHA, Tokyo (JP)

(72) Inventor: Naoki IIDA, Fujinomiya-shi (JP)

(21) Appl. No.: 19/420,209

(22) Filed: Dec. 15, 2025

Related U.S. Application Data
(63) Continuation of application No. PCT/JP2024/021795, filed on Jun. 17, 2024.

(30) Foreign Application Priority Data
Jun. 16, 2023 (JP) 2023099351

(51) Int. Cl. A61M 1/02

(52) U.S. Cl. CPC A61M 1/0281; A61M 2202/0028; A61M 2202/0439; A61M 2205/3331; A61M 2205/7545; A61M 2206/12

(57) ABSTRACT

A filter device according to the present invention is to be mounted on a centrifugal separator and includes: a first space into which blood flows; a second space from which the blood flows out; a partition wall separating the first space and the second space; a communication port establishing communication between the first space and the second space; and a white-blood-cell removal filter disposed in the second space. The second space is located between the first space and a rotation center of the centrifugal separator.

### CROSS-REFERENCE TO RELATED APPLICATIONS

The present disclosure is a continuation of and claims benefit to PCT/JP2024/021795 filed on Jun. 17, 2024, entitled “FILTER DEVICE” which claims priority to Japanese Patent Application No. 2023-099351, filed on Jun. 16, 2023. The entire disclosure of the applications listed above are hereby incorporated herein by reference, in their entireties, for all that they teach and for all purposes.

### BACKGROUND

The present disclosure relates to a filter device.

Japanese Patent No. 5223006 B2 describes a blood bag system and a centrifugal transfer device (e.g., centrifugal separator). The blood bag system accommodates blood. The centrifugal separator centrifuges the blood accommodated in the blood bag system. The centrifuged blood is used for blood transfusion.

### BRIEF SUMMARY

It is preferable that blood to be transfused does not contain white blood cells. Recently, a filter device capable of more suitably removing white blood cells is desired.

It is an object of the present disclosure to solve the problem described above.

One aspect of the present disclosure is (1) a filter device to be mounted on a centrifugal separator, the filter device including: a first space which is formed in a housing and into which blood flows through an inlet port; a second space which is formed in the housing and from which the blood flows out through an outlet port; a partition wall that separates the first space and the second space; a communication port that establishes communication between the first space and the second space; and a white-blood-cell removal filter that is disposed in the second space and that removes a white blood cell contained in the blood, wherein the second space is located between the first space and a rotation center of the centrifugal separator.

With this configuration, white blood cells can be suitably removed from blood.

Aspect (2) describes the filter device according to aspect (1), in which the inlet port may be located on one side of the first space in a first direction that intersects a centrifugal direction in which a centrifugal force is applied, and the communication port may be located on another side of the first space in the first direction.

This configuration makes it possible to prevent blood from which white blood cells have not been removed by centrifugation from reaching the second space.

Aspect (3) describes the filter device according to aspect (1) or aspect (2), in which the outlet port may be located on one side of the second space in a first direction, and the communication port may be located on another side of the second space in the first direction.

This configuration makes it possible to more reliably remove white blood cells by the white-blood-cell removal filter.

Aspect (4) describes the filter device according to any one of aspects (1) to (3) provided above, in which the first space may include a guide wall that guides the blood flowing in through the inlet port in a centrifugal direction in which a centrifugal force is applied.

This configuration makes it possible to prevent blood from which white blood cells have not been removed by centrifugation from reaching the second space.

Aspect (5) describes the filter device according to aspect (4), in which the inlet port and the communication port may be located on one side of the first space in a first direction intersecting a centrifugal direction in which a centrifugal force is applied.

This configuration makes it possible to further prevent blood from which white blood cells have not been removed by centrifugation from reaching the second space.

Aspect (6) describes the filter device according to aspect (5), in which the communication port may be located on one side of the second space in the first direction, and the outlet port may be located on another side of the second space in the first direction.

This configuration makes it possible to more reliably remove white blood cells by the white-blood-cell removal filter.

Aspect (7) describes the filter device according to any one of aspects (1) to (6) provided above, in which the first space may include a curved wall, and the inlet port and the communication port may be located between a rotation center of the centrifugal separator and the curved wall.

This configuration makes it possible to prevent backflow of blood toward the inlet port.

Aspect (8) describes the filter device according to any one of aspects (1) to (7) provided above, in which the housing may be provided with an inflow channel that reaches the first space from the inlet port and that is separated from the second space, and the inflow channel may extend along a centrifugal direction in which a centrifugal force is applied.

With this configuration, the blood can smoothly flow into the first space while being applied with a centrifugal force.

One aspect of the present disclosure is (9) a filter device, comprising: a housing, comprising: a first interior space formed inside the housing, the first interior space comprising an inlet port arranged in fluid communication with the first interior space and a curved interior wall arranged opposite the inlet port; a second interior space formed inside the housing, the second interior space comprising an outlet port arranged in fluid communication with the second interior space; a partition wall arranged between the first interior space and the second interior space, wherein the partition wall separates the first interior space from the second interior space; and a communication port disposed in the partition wall, the communication port arranged in fluid communication with the first interior space and the second interior space; wherein a centrifugal blood flow path is defined by the housing as extending from the inlet port in a centrifugal direction toward the curved interior wall, following around the curved interior wall in the first interior space and then in a centripetal direction toward the communication port, through the communication port and into the second interior space, and out of the outlet port in the centripetal direction, wherein the centripetal direction is arranged opposite the centrifugal direction.

Aspect (10) describes the filter device according to aspect (9), in which the filter device further comprises: a white-blood-cell removal filter disposed in the second interior space, wherein the white-blood-cell removal filter comprises a filter medium that is configured to remove white blood cells from a portion of blood flowing through the white-blood-cell removal filter. Aspect (11) describes the filter device according to aspect (9) or aspect (10), in which the housing further comprises: an inflow channel extending inside the housing between the inlet port and the first interior space, wherein the inflow channel extends a distance in the centrifugal direction. Aspect (12) describes the filter device according to any one of aspects (9) to (11), in which the first interior space comprises: a first section arranged adjacent the inflow channel; and a second section arranged adjacent the communication port, wherein the curved interior wall follows an arch extending inside the first interior space between the first section and the second section. Aspect (13) describes the filter device according to any one of aspects (9) to (12), in which the inlet port is arranged extending through a base wall section of the housing into the inflow channel, wherein the first interior space is arranged a first distance away from the base wall section of the housing, wherein the second interior space is arranged a second distance away from the base wall section of the housing, and wherein the second distance is less than the first distance. Aspect (14) describes the filter device according to any one of aspects (9) to (13), in which a center line of the first interior space is arranged parallel to a center line of the second interior space. Aspect (15) describes the filter device according to any one of aspects (9) to (14), in which the center line of the first interior space is offset by an offset distance from the center line of the second interior space measured along a first direction that is perpendicular to the centrifugal direction. Aspect (16) describes the filter device according to any one of aspects (9) to (15), in which the first section of the first interior space is arranged on a first side of the center line of the first interior space, wherein the second section of the first interior space is arranged on a second side of the center line of the first interior space, and wherein the second side is opposite the first side. Aspect (17) describes the filter device according to any one of aspects (9) to (16), in which the communication port is arranged extending from a point on the second side of the center line of the first interior space into the second interior space. Aspect (18) describes the filter device according to any one of aspects (9) to (17), in which the outlet port is arranged extending through the base wall section of the housing into the second interior space. Aspect (19) describes the filter device according to any one of aspects (9) to (18), in which an inflow direction defined by the inlet port is arranged extending in the centrifugal direction, wherein an outflow direction defined by the outlet port is arranged extending in the centripetal direction, and wherein the inflow direction and the outflow direction are arranged parallel to one another.

One aspect of the present disclosure is (20) a filter device, comprising: a housing, comprising: a first interior space formed inside the housing, the first interior space comprising an inlet port arranged in fluid communication with the first interior space and a curved interior wall arranged opposite the inlet port; a second interior space formed inside the housing, the second interior space comprising an outlet port arranged in fluid communication with the second interior space; a partition wall arranged between the first interior space and the second interior space, wherein the partition wall separates the first interior space from the second interior space; a communication port disposed in the partition wall, the communication port arranged in fluid communication with the first interior space and the second interior space; and a white-blood-cell removal filter disposed in the second interior space, wherein the white-blood-cell removal filter comprises a filter medium that is configured to remove white blood cells from a portion of blood flowing through the white-blood-cell removal filter; wherein a centrifugal blood flow path is defined by the housing as extending from the inlet port in a centrifugal direction toward the curved interior wall, following around the curved interior wall in the first interior space and then in a centripetal direction toward the communication port, through the communication port and into the second interior space, and out of the outlet port in the centripetal direction, wherein the centripetal direction is arranged opposite the centrifugal direction.

According to the present disclosure, it is possible to suitably remove white blood cells from blood.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages are described herein and will be apparent to those skilled in the art upon consideration of the following Detailed Description and in view of the figures.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a perspective view illustrating a centrifugal separator to which a filter device according to embodiments of the present disclosure is mounted.

FIG. 2 is a perspective view illustrating the filter device in accordance with embodiments of the present disclosure.

FIG. 3 is a plan view illustrating an internal structure of the filter device in accordance with embodiments of the present disclosure.

FIG. 4 is a plan view illustrating an internal structure of a filter device according to a modification.

### DETAILED DESCRIPTION

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

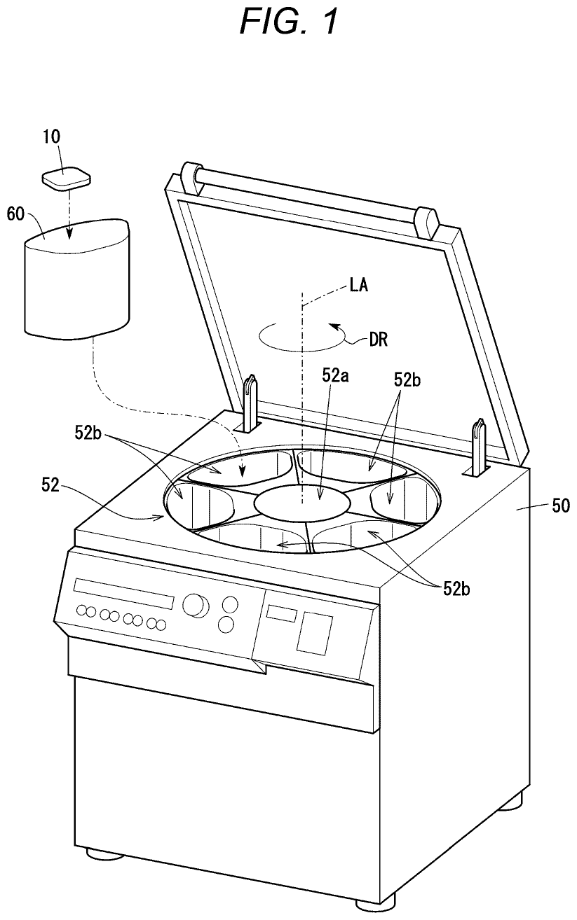

A filter device according to at least one embodiment of the present disclosure will be described with reference to the drawings. FIG. 1 is a perspective view illustrating a centrifugal separator 50 to which a filter device 10 according to a first embodiment is to be mounted. FIG. 1 illustrates the filter device 10, an insert unit 60, and a centrifugal separator 50.

The centrifugal separator 50 is a machine that centrifugally separates blood. The centrifugal separator 50 includes a centrifugal drum 52. The centrifugal drum 52 includes a central body 52a and a plurality of unit insertion portions 52b (e.g., receivers).

The plurality of unit insertion portions 52b are disposed so as to surround the central body 52a. An insert unit 60 (e.g., container, receptacle, etc.) can be inserted into each of the plurality of unit insertion portions 52b. The insert unit 60 is attached to the centrifugal separator 50 by being inserted into the corresponding unit insertion portion 52b.

A blood bag system (not illustrated) may be accommodated in the insert unit 60. The blood bag system may include a blood bag containing blood before being centrifuged. The blood contained in the blood bag is, for example, whole blood, but may be buffy coat as described in Japanese Patent No. 5223006 B2 above. The buffy coat contains red blood cells, platelets, white blood cells, and the like.

The centrifugal separator 50 rotates the insert unit 60 inserted into the unit insertion portion 52b about the central body 52a. More specifically, the centrifugal separator 50 rotates the insert unit 60 inserted into the unit insertion portion 52b along a rotation direction DR about the center line of rotation (e.g., rotation center) LA illustrated in FIG. 1. Accordingly, a centrifugal force is applied to the entire insert unit 60. The blood is centrifuged by the centrifugal force. The center line of rotation LA is, for example, along a gravity direction (e.g., second direction D2 to be described later).

The filter device 10 is mounted on the insert unit 60. The filter device 10 is mounted to, for example, but not limited to, an upper part of the insert unit 60.

The filter device 10 is mounted to the insert unit 60, and thus is mounted to the centrifugal separator 50 via the insert unit 60. The centrifugal force described above is applied not only to the insert unit 60 but also to the filter device 10. Note that the filter device 10 may be directly mounted to the centrifugal separator 50.

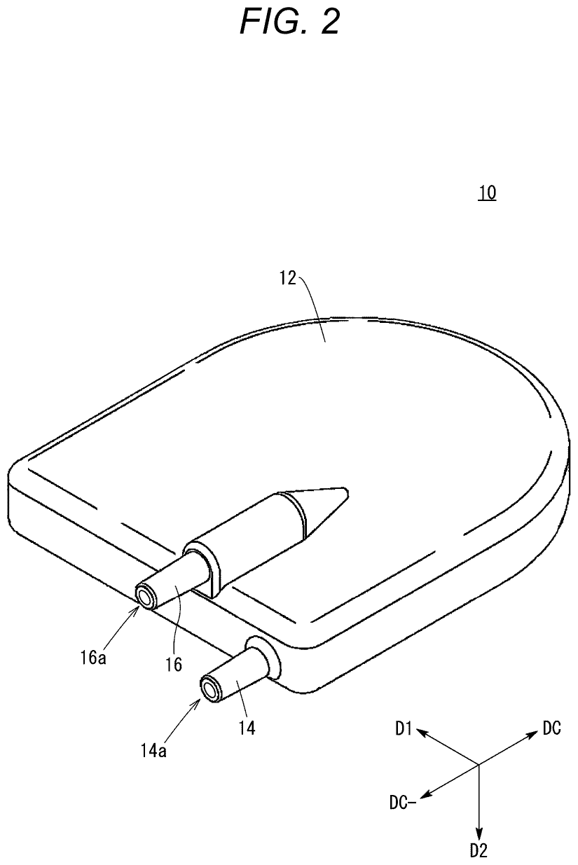

FIG. 2 is a perspective view illustrating the filter device 10 in accordance with embodiments of the present disclosure.

As described above, the filter device 10 may be mounted to the centrifugal separator 50. In the following, the configuration of the filter device 10 will be described based on the premise that the filter device 10 is mounted to the centrifugal separator 50.

As illustrated in FIG. 2, reference may be made to a centrifugal direction DC, a centripetal direction DC−, a first direction D1, and a second direction D2. The centrifugal direction DC is a direction of the centrifugal force applied to the filter device 10 by the centrifugal separator 50. The first direction D1 is a direction of a tangential velocity in the circular movement of the filter device 10 by the centrifugal separator 50. The first direction D1 is orthogonal to (e.g., intersects) the centrifugal direction DC. In some embodiments, the first direction D1 may be arranged perpendicular to the centrifugal direction DC. The second direction D2 is a gravity direction. In the present embodiment, the centrifugal direction DC and the first direction D1 are orthogonal to the second direction D2. The centripetal direction DC− is a direction opposite to the centrifugal direction DC.

The filter device 10 includes a housing 12. The housing 12 can be formed in, for example, a box shape. In the present embodiment, the thickness direction of the housing 12 coincides with the second direction D2. Stated another way, the thickness of the housing 12 may be measured as a dimension taken along the second direction D2. A length of the housing 12 may be measured as a dimension in the first direction D1, and a width of the housing 12 may be measured as a dimension in the centrifugal direction DC and/or centripetal direction DC−.

The housing 12 includes an inlet portion 14 and an outlet portion 16. The inlet portion 14 has an inlet port 14a. The outlet portion 16 has an outlet port 16a. Each of the inlet port 14a and the outlet port 16a is an opening that establishes communication between the inside and the outside of the housing 12. Stated another way, the inlet port 14a is fluidly connected to the outlet port 16a via at least one passage inside the housing 12. The inlet portion 14 and the outlet portion 16 illustrated in FIG. 2 protrude from the housing 12, but are not limited thereto.

Different blood bags may be connected to the inlet portion 14 and the outlet portion 16. The blood bag connected to the inlet portion 14 is a blood bag containing, in advance, blood before being centrifuged. The blood bag connected to the outlet portion 16 is a blood bag for accommodating blood (e.g., a blood component) that has passed through the filter device 10.

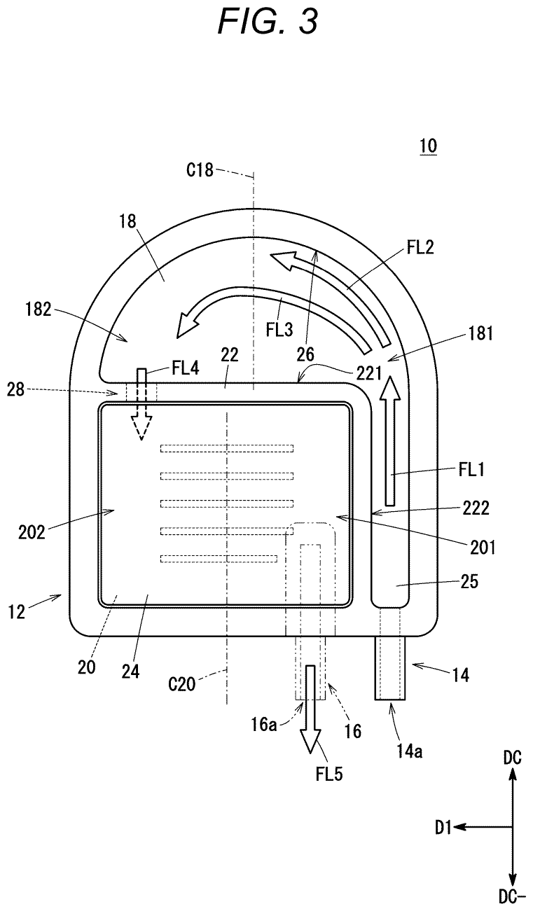

FIG. 3 is a plan view illustrating an internal structure of the filter device 10 in accordance with embodiments of the present disclosure.

The housing 12 further includes an inflow channel 25, a first space 18 (e.g., first interior space), a second space 20 (e.g., a second interior space), a partition wall 22, a white-blood-cell removal filter 24, and a communication port 28. The inflow channel 25, the first space 18, the second space 20, the partition wall 22, and the communication port 28 are provided in the housing 12. The white-blood-cell removal filter 24 is disposed in the second space 20. The first space 18 may be fluidly interconnected to the second space 20 by the communication port 28, which passes through the partition wall 22.

The first space 18 is located in the centrifugal direction DC relative to the inflow channel 25 and the second space 20. In other words, the inflow channel 25 and the second space 20 are located between the first space 18 and the rotation center LA (see, e.g., FIG. 1) of the centrifugal separator 50. The second space 20 is located in the first direction D1 relative to the inflow channel 25.

The partition wall 22 separates the first space 18 and the second space 20, and separates the second space 20 and the inflow channel 25. More specifically, the partition wall 22 has a first wall portion 221 along the first direction D1 and a second wall portion 222 along the centrifugal direction DC. The first space 18 and the second space 20 are separated by the first wall portion 221. The second space 20 and the inflow channel 25 are separated by the second wall portion 222.

The inflow channel 25 is a flow path reaching the first space 18 from the inlet port 14a. The inflow channel 25 extends along the centrifugal direction DC. Blood flows into the inflow channel 25 through the inlet port 14a.

Note that the inlet port 14a may be formed along the centrifugal direction DC. In this case, the blood smoothly flows through the inlet port 14a and flows into the first space 18 by the centrifugal force applied by the centrifugal separator 50.

The first space 18 has a first section 181 and a second section 182. The first section 181 is located on one side relative to a center line C18. The second section 182 is located on the other side relative to the center line C18. The center line C18 is a virtual straight line passing through the center of the first space 18 in the first direction D1 and extending along the centrifugal direction DC. The second section 182 is located in the first direction D1 relative to the first section 181.

The first section 181 of the first space 18 is connected to the inflow channel 25. The blood can flow into the first section 181 of the first space 18 along the inflow channel 25 (FL1).

The first space 18 has a curved wall 26. The curved wall 26 is a part of the inner wall defining the first space 18. The inflow channel 25 (e.g., fluidly interconnected to the inlet port 14a) and the communication port 28 are located between the rotation center LA (see, e.g., FIG. 1) of the centrifugal separator 50 and the curved wall 26.

The curved wall 26 is curved to guide blood from the first section 181 toward the second section 182. Thus, the blood flowing into the first section 181 through the inflow channel 25 can flow toward the second section 182 along the curved wall 26 without flowing back toward the inlet port 14a (FL2, FL3).

The blood flowing into the first space 18 is centrifuged by the centrifugal separator 50. As a result, the blood in the first space 18 has a layer of a supernatant liquid and a layer of a sedimentation liquid. The layer of the sedimentation liquid is located in the centrifugal direction DC relative to the layer of the supernatant liquid.

The main component of the sedimentation liquid is white blood cells. The reason why white blood cells are considered to be the main component of the sedimentation liquid is that white blood cells are relatively heavy blood components. That is, white blood cells are blood components that are relatively easily precipitated, and thus, are main components of the sedimentation liquid. On the other hand, the supernatant liquid contains a blood component lighter than white blood cells. For example, the supernatant liquid contains large quantities of platelets and the like.

In this manner, the white blood cells contained in the blood flowing into the first space 18 are collected on the side of the first space 18 in the centrifugal direction DC by the centrifugal force applied by the centrifugal separator 50 (FL2). On the other hand, blood components other than white blood cells are collected on the side of the first space 18 in the centripetal direction DC− (FL3).

The communication port 28 is an opening that establishes communication (e.g., fluid communication, etc.) between the first space 18 and the second space 20. The communication port 28 is located between the first space 18 and the second space 20. As illustrated in FIG. 3, the second space 20 is located in the centripetal direction DC− relative to the first space 18. Therefore, the communication port 28 is located in the centripetal direction DC− relative to the first space 18. The communication port 28 is formed in the first wall portion 221 of the partition wall 22, for example, but is not limited thereto.

At least a portion of the blood flowing into the first space 18 can flow into the second space 20 through the communication port 28 (FL4). As described above, blood components other than white blood cells are collected on the side of the first space 18 in the centripetal direction DC−. Therefore, blood components other than white blood cells easily flow into the second space 20 through the communication port 28. In other words, white blood cells are less likely to flow into the second space 20 than the other components in the blood.

As described above, the inlet port 14a (e.g., and the inflow channel 25) is connected to the first section 181 of the first space 18. In such a case, the communication port 28 is preferably connected to the second section 182 of the first space 18. With this configuration, it is possible to prevent the blood flowing by the centrifugal force from immediately flowing from the inlet port 14a to the communication port 28. That is, it is possible to prevent the blood flowing in through the inlet port 14a from reaching the communication port 28 before being separated into the above-described sedimentation liquid and supernatant liquid.

As described above, the white-blood-cell removal filter 24 is disposed in the second space 20. The white-blood-cell removal filter 24 includes a filter medium for removing white blood cells from blood. The blood that flows into the second space 20 is filtered by the white-blood-cell removal filter 24. Thus, even if white blood cells flow into the second space 20, the white blood cells are removed by the white-blood-cell removal filter 24.

The second space 20 is provided with the outlet portion 16. The outlet portion 16 is provided with the outlet port 16a. The outlet port 16a is in fluid communication with the second space 20. Therefore, the blood (e.g., blood without white blood cells, etc.) in the second space 20 can flow out of the housing 12 through the outlet port 16a (FL5).

Moreover, the outlet port 16a is located between the communication port 28 and the rotation center LA (FIG. 1) of the centrifugal separator 50. With this configuration, it is possible to prevent the white blood cells flowing into the second space 20 from reaching the outlet port 16a. That is, in order for the blood flowing into the second space 20 to reach the outlet port 16a, the blood may need to move in the centripetal direction DC− from the communication port 28. As described above, the white blood cells are less likely to flow in the centripetal direction DC− during the centrifugation with the centrifugal separator 50. Since the direction from the outlet port 16a toward the communication port 28 is the centrifugal direction DC as illustrated in FIG. 3, it is possible to prevent white blood cells from reaching the outlet port 16a. Thus, the amount of white blood cells contained in the blood taken out from the outlet port 16a can be more suitably reduced.

The second space 20 includes a first section 201 and a second section 202. The first section 201 is located on one side relative to a center line C20. The second section 202 is located on the other side relative to the center line C20. The center line C20 is a virtual straight line passing through the center of the second space 20 in the first direction D1 and extending along the centrifugal direction DC. The second section 202 is located in the first direction D1 relative to the first section 201.

The communication port 28 is connected to the second section 202 of the second space 20. In such a case, the outlet port 16a is preferably connected to the first section 201 of the second space 20. With this configuration, it is possible to obtain a sufficiently long movement path of blood in the white-blood-cell removal filter 24. This configuration makes it possible to more reliably remove white blood cells by the white-blood-cell removal filter 24.

As described above, according to the present embodiment, the blood flowing into the first space 18 is centrifuged by the centrifugal separator 50. Thus, the amount of white blood cells contained in the blood flowing into the second space 20 is reduced. The white blood cells remaining in the blood flowing into the second space 20 are removed by the white-blood-cell removal filter 24. That is, according to the present embodiment, the removal of white blood cells by centrifugation and the removal of white blood cells by the white-blood-cell removal filter 24 can be achieved on blood only by operating the centrifugal separator 50.

Modifications according to the above embodiment will be described below. Note that the description overlapping with the above embodiment will be appropriately omitted. Elements described in the above embodiment are denoted by the same reference numerals as those in the above embodiment unless otherwise specified.

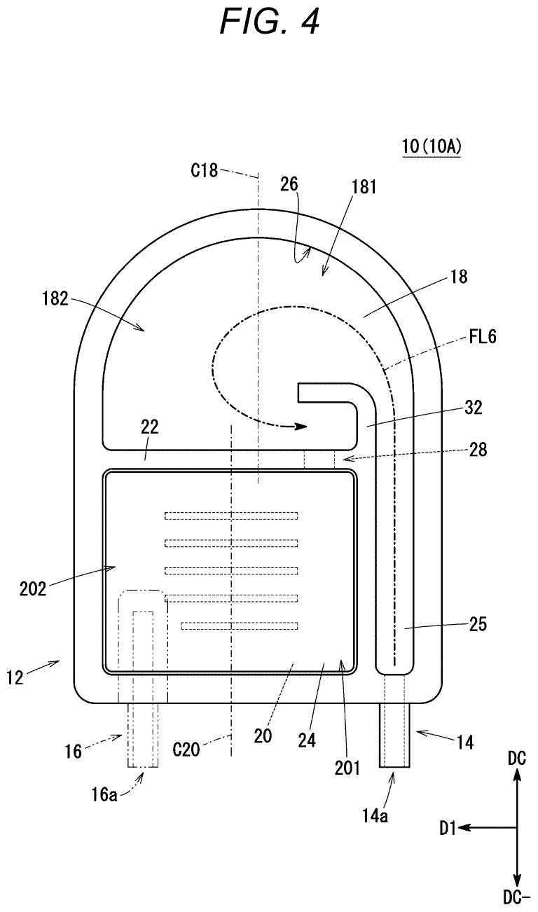

FIG. 4 is a plan view illustrating an internal structure of a filter device 10 (10A) according to a first modification.

A first space 18 may be provided with a guide wall 32 that guides blood flowing in through an inlet port 14a in the centrifugal direction DC. The guide wall 32 is positioned between a communication port 28 and the inlet port 14a (e.g., and inflow channel 25) in the first direction D1. The guide wall 32 extends, for example, in the centrifugal direction DC, and further extends in the first direction D1.

The guide wall 32 inhibits the blood flowing into the first space 18 from immediately reaching the communication port 28 along the first direction D1 (FL6). That is, the blood flowing into the first space 18 from the inlet port 14a cannot reach the communication port 28 unless the blood bypasses the guide wall 32. Thus, it is possible to prevent the blood before being centrifuged in the first space 18 from immediately reaching the communication port 28.

When the guide wall 32 is provided in the first space 18, the communication port 28 may be located in a first section 181 of the first space 18. This is because, as described above, the guide wall 32 can inhibit the blood flowing into the first space 18 from immediately reaching the communication port 28 along the first direction D1.

Note that the communication port 28 located in the first section 181 of the first space 18 can be connected to a first section 201 of a second space 20. In such a case, it is preferable to position an outlet port 16a in a second section 202 of the second space 20. With this configuration, it is possible to obtain a sufficiently long movement path of blood in the white-blood-cell removal filter 24. This configuration makes it possible to more reliably remove white blood cells by the white-blood-cell removal filter 24.

Note that the present invention is not limited to the above disclosure and can take various configurations without departing from the gist of the present invention.

References in the specification to “one embodiment,” “an embodiment,” “an example embodiment,” “some embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in conjunction with one embodiment, it is submitted that the description of such feature, structure, or characteristic may apply to any other embodiment unless so stated and/or except as will be readily apparent to one skilled in the art from the description. The present disclosure, in various embodiments, configurations, and aspects, includes components, methods, processes, systems and/or apparatus substantially as depicted and described herein, including various embodiments, subcombinations, and subsets thereof. Those of skill in the art will understand how to make and use the systems and methods disclosed herein after understanding the present disclosure. The present disclosure, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease, and/or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments, configurations, or aspects for the purpose of streamlining the disclosure. The features of the embodiments, configurations, or aspects of the disclosure may be combined in alternate embodiments, configurations, or aspects other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the disclosure.

Moreover, though the description of the disclosure has included description of one or more embodiments, configurations, or aspects and certain variations and modifications, other variations, combinations, and modifications are within the scope of the disclosure, e.g., as may be within the skill and knowledge of those in the art, after understanding the present disclosure. It is intended to obtain rights, which include alternative embodiments, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

It is to be appreciated that any feature described herein can be claimed in combination with any other feature(s) as described herein, regardless of whether the features come from the same described embodiment.

As used herein, the singular forms “a,” “an,” and “the” are intended to include the plural forms as well, unless the context clearly indicates otherwise. It will be further understood that the terms “include,” “including,” “includes,” “comprise,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. The term “and/or” includes any and all combinations of one or more of the associated listed items.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B, and C together. When each one of A, B, and C in the above expressions refers to an element, such as X, Y, and Z, or a class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and this disclosure.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

What is claimed is:

1. A filter device to be mounted on a centrifugal separator, the filter device comprising:
a first space which is formed in a housing and into which blood flows through an inlet port;
a second space which is formed in the housing and from which the blood flows out through an outlet port;
a partition wall that separates the first space and the second space;
a communication port that establishes communication between the first space and the second space; and
a white-blood-cell removal filter that is disposed in the second space and that removes a white blood cell contained in the blood, wherein the second space is located between the first space and a rotation center of the centrifugal separator.

2. The filter device of claim 1, wherein the inlet port is located on one side of the first space in a first direction that intersects a centrifugal direction in which a centrifugal force is applied, and wherein the communication port is located on another side of the first space in the first direction.

3. The filter device of claim 2, wherein the outlet port is located on one side of the second space in the first direction, and wherein the communication port is located on another side of the second space in the first direction.

4. The filter device of claim 1, wherein the first space includes a guide wall that guides the blood flowing in through the inlet port in a centrifugal direction in which a centrifugal force is applied.

5. The filter device of claim 4, wherein the inlet port and the communication port are located on one side of the first space in a first direction intersecting a centrifugal direction in which a centrifugal force is applied.

6. The filter device of claim 5, wherein the communication port is located on one side of the second space in the first direction, and wherein the outlet port is located on another side of the second space in the first direction.

7. The filter device of claim 1, wherein the first space includes a curved wall, and wherein the inlet port and the communication port are located between a rotation center of the centrifugal separator and the curved wall.

8. The filter device of claim 1, wherein the housing is provided with an inflow channel that reaches the first space from the inlet port and that is separated from the second space, and wherein the inflow channel extends along a centrifugal direction in which a centrifugal force is applied.

9. A filter device, comprising:
a housing, comprising:
a first interior space formed inside the housing, the first interior space comprising an inlet port arranged in fluid communication with the first interior space and a curved interior wall arranged opposite the inlet port;
a second interior space formed inside the housing, the second interior space comprising an outlet port arranged in fluid communication with the second interior space;
a partition wall arranged between the first interior space and the second interior space, wherein the partition wall separates the first interior space from the second interior space; and
a communication port disposed in the partition wall, the communication port arranged in fluid communication with the first interior space and the second interior space;
wherein a centrifugal blood flow path is defined by the housing as extending from the inlet port in a centrifugal direction toward the curved interior wall, following around the curved interior wall in the first interior space and then in a centripetal direction toward the communication port, through the communication port and into the second interior space, and out of the outlet port in the centripetal direction, wherein the centripetal direction is arranged opposite the centrifugal direction.

10. The filter device according to claim 9, further comprising:
a white-blood-cell removal filter disposed in the second interior space, wherein the white-blood-cell removal filter comprises a filter medium that is configured to remove white blood cells from a portion of blood flowing through the white-blood-cell removal filter.

11. The filter device of claim 10, wherein the housing further comprises:
an inflow channel extending inside the housing between the inlet port and the first interior space, wherein the inflow channel extends a distance in the centrifugal direction.

12. The filter device of claim 11, wherein the first interior space comprises:
a first section arranged adjacent the inflow channel; and
a second section arranged adjacent the communication port, wherein the curved interior wall follows an arch extending inside the first interior space between the first section and the second section.

13. The filter device of claim 12, wherein the inlet port is arranged extending through a base wall section of the housing into the inflow channel, wherein the first interior space is arranged a first distance away from the base wall section of the housing, wherein the second interior space is arranged a second distance away from the base wall section of the housing, and wherein the second distance is less than the first distance.

14. The filter device of claim 13, wherein a center line of the first interior space is arranged parallel to a center line of the second interior space.

15. The filter device of claim 14, wherein the center line of the first interior space is offset by an offset distance from the center line of the second interior space measured along a first direction that is perpendicular to the centrifugal direction.

16. The filter device of claim 15, wherein the first section of the first interior space is arranged on a first side of the center line of the first interior space, wherein the second section of the first interior space is arranged on a second side of the center line of the first interior space, and wherein the second side is opposite the first side.

17. The filter device of claim 16, wherein the communication port is arranged extending from a point on the second side of the center line of the first interior space into the second interior space.

18. The filter device of claim 17, wherein the outlet port is arranged extending through the base wall section of the housing into the second interior space.

19. The filter device of claim 18, wherein an inflow direction defined by the inlet port is arranged extending in the centrifugal direction, wherein an outflow direction defined by the outlet port is arranged extending in the centripetal direction, and wherein the inflow direction and the outflow direction are arranged parallel to one another.

20. A filter device, comprising:
a housing, comprising:
a first interior space formed inside the housing, the first interior space comprising an inlet port arranged in fluid communication with the first interior space and a curved interior wall arranged opposite the inlet port;
a second interior space formed inside the housing, the second interior space comprising an outlet port arranged in fluid communication with the second interior space;
a partition wall arranged between the first interior space and the second interior space, wherein the partition wall separates the first interior space from the second interior space;
a communication port disposed in the partition wall, the communication port arranged in fluid communication with the first interior space and the second interior space; and
a white-blood-cell removal filter disposed in the second interior space, wherein the white-blood-cell removal filter comprises a filter medium that is configured to remove white blood cells from a portion of blood flowing through the white-blood-cell removal filter;
wherein a centrifugal blood flow path is defined by the housing as extending from the inlet port in a centrifugal direction toward the curved interior wall, following around the curved interior wall in the first interior space and then in a centripetal direction toward the communication port, through the communication port and into the second interior space, and out of the outlet port in the centripetal direction, wherein the centripetal direction is arranged opposite the centrifugal direction.
